(19) United States
(12) Patent Application Publication
(10) Pub. No.: US 2005/0221475 A1
(43) Pub. Date: Oct. 6, 2005

(54) Cell culture procedures for inducing a neuronal phenotype in insect cells

(76) Inventors: Jeffrey Richard Bloomquist, Blacksburg, VA (US); Sally Louise Paulson, Blacksburg, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/097,206

(22) Filed: Apr. 4, 2005

Related U.S. Application Data
(60) Provisional application No. 60/559,007, filed on Apr. 5, 2004.

(51) Int. Cl. C12N 5/06; A01N 45/00

(52) U.S. Cl. 435/348; 514/169

(57) ABSTRACT

A method for inducing non-neuronal insect cells cultured in vitro to exhibit a neuronal phenotype is provided. The method involves exposing the non-neuronal cells to a transcription regulating agent such as a steroidal insect hormone (e.g. 20-hydroxyecdysone), and, optionally, to a mild depolarizing agent. The induced cells express proteins specific for neuronal cells (e.g. ion channels and neurotransmitter receptors), and may also exhibit morphological changes characteristic of neuronal cells (e.g. development of axons and synapses).

This application claims benefit of U.S. provisional patent application 60/559,007, filed Apr. 5, 2004, the complete contents of which are hereby incorporated by reference.

### BACKGROUND OF THE INVENTION

1. Field of the Invention

The invention generally relates to a method for inducing non-neuronal insect cells cultured in vitro to exhibit a neuronal phenotype, including the expression of neuron specific proteins and neuron associated morphological changes. In particular, the invention provides a method for inducing a neuronal phenotype in insect cells cultured in vitro by exposing the cells to a steroidal insect hormone and, optionally, a mild depolarizing agent.

2. Background of the Invention

A significant problem in the attempts to discover new insecticides, nematicides, or drugs that effect insects or insect cells is the procurement of large amounts of insect nervous tissue for high throughput chemical screening. At present, all in vitro work with insect nervous tissue employs primary cultures that are time-consuming to establish and typically survive only a few weeks at most.

Work undertaken in the area includes the following:
1) Decombel et al. (2004) describes effects of insecticides on continuous cell lines of the Beet Armyworm, Spodoptera exigua. The neurotoxic compounds tested showed little effect in the cell proliferation assay that was used. This is not surprising since neuroreceptor/ion channel expression was absent in these undifferentiated cells, especially the voltage-sensitive sodium channels that comprise the major target site for the pyrethroid insecticides, such as bifenthrin, which was of low activity in these cells.
2) Grünewald and Levine (1998) showed that the insect hormone 20-hydroxyecdysone increases calcium currents in pupal leg motoneurons, when the cells are dissected and maintained in primary culture. The techniques employed in the study included hand dissections of nervous tissue that were highly labor intensive. In addition, 20-hydroxyecdysone did not affect cell morphology in these studies.
3) Wegener et al. (1996) discuss a fly cell line in which constitutive expression of muscarinic-type acetylcholine receptors was increased by 20-hydroxyecdysone. No sodium channel expression or synaptogenesis was reported. The insect sodium channel is common within the insect nervous system and is known to be involved in insecticide action (Decombel et al., (2004)). Its absence indicates no general expression of neuronal phenotype in the cells that were studied, because unlike mammals, sodium channels in insects are confined to the nervous system (Bloomquist, 1992; Song et al., 2004).
4) Kislev et al. (1984) report morphological changes in SF-21 cells in response to 20-hydroxyecdysone. No synapse-like structures were noted in the cultures and there is no mention of receptor/ion channel expression or insecticide screening in this report.
5) Lynn and Oberlander (1981) showed that 20-Hydoxyecdysone caused cell elongation that was blocked by cytoskeletal disrupting agents. Again, no functional protein expression or synaptic structures were identified in the course of the work.

The next five papers pertain to the induced expression of acetylcholinesterase in mosquito or Drosophila cell lines. The original observation is that of Cherbas et al. (1977) who showed that 20-hydroxyecdysone induced the expression of acetylcholinesterase in Kc-H cells. A similar effect was observed in later studies (Best-Belpomme et al., 1980; Berger and Wyss, 1980). The enzyme is typically indistinguishable in character from brain acetylcholinesterase, but found at lower levels. Cherbas et al., (1977) hypothesized that the Kc-H cells may be precursors of neurons or glial elements, but concluded that additional markers were necessary to establish this fact. More recent studies by Andres and Cherbas (1994) found that the Kc cells are of hematopoietic, not neuronal origin. The paper by Cohen (1980) reports a similar increase in acetylcholinesterase activity in mosquito cells by adding 20-hydroxyecdysone to the medium, and high background levels of enzyme activity are maintained by the fetal calf serum. However, there is no particular advantage in being able to express acetylcholinesterase in a cell line, because this enzyme is easily harvested in large amounts from frozen insect tissue for insecticide screening efforts (Camp et al., (1969)).
6) Andres and Cherbas (1994) investigated the Eip28/29 gene of Drosophila as an example of a tissue- and stage-specific ecdysone-responsive gene, studying the expression (in transgenic flies) of reporter genes controlled by Eip28/29-derived flanking DNA. They found that during the middle and late third instar, most tissues exhibit normal expression patterns when controlled by one of two classes of regulatory sequences: 1) Class A sequences include only 657 Np of 5′ flanking DNA from Eip28/29; and 2) Class B sequences include an extended 3′ flanking region and a minimal (less than or equal to 93 Np) 5′ flanking region. The class B sequences include all those elements known to be important for ecdysone induction in cultured cells, and are sufficient to direct the normal premetamorphic induction of Eip28/29 in the lymph glands, hemocytes, proventriculus, and Malpighian tubules. Neuron-specific induction was not observed.
7) Cohen (1981) discusses the detection of acetylcholinesterase (ACHE) activity in an Aedes aegypti established cell line. The enzyme is blocked by 10⁻⁶ M serine sulfate, displays excess substrate inhibition and slowly hydrolyzes butyrylthiocholine. A 2-fold stimulation of AChE activity was shown after 2 days exposure to 3×10⁻⁷ M beta-ecdysone. AChE activity found in the fresh medium is the contribution of the fetal calf serum portion. A direct relationship between levels of serum and the AChE activity in the cultured cells was demonstrated.
8) Best-Belpomme et al. (1980) discuss results on Kc cell expression of acetylcholinesterase that are similar to results obtained by Cherbas et al., (1977).
9) Berger and Wyss (1980) show that Drosophila S3 line cells maintain a high basal level of acetylcholinesterase (AChE) activity, which is lost and then reinduced following exposure to the steroid, beta-ecdysone. Electrophoretic studies indicate that the basal and reinduced AChE activities may be different isozymes. In contrast, MDR line cells have a low basal AChE activity which is induced to a modest level by hormone. In S3/MDR hybrids the high basal level phenotype is extinguished, and the hormone-induced AChE level is modest. MDER line cells, which continue to grow in the presence of beta-ecdysone and are therefore termed resistant, show a low basal AChE level that is not further elevated by hormone. This phenotype and response also occurred in beta-ecdysone-resistant S3/MDER hybrids.
9) Cherbas et al. (1977) show that when cells of the Drosophila Kc-H line are treated with greater than or equal to 10⁻⁸ molar beta-ecdysone, they extend long processes and acquire acetylcholinesterase activity.

Although each of the cited publications provide data concerning insect cells in culture, some of which explores effects of 20HE, none provides methods for inducing cultured insect cells to exhibit a neuronal phenotype involving the expression of ion channels such as the sodium channel.

### SUMMARY OF THE INVENTION

It is an object of this invention to provide a method of inducing non-neuronal insect cells that are cultured in vitro to exhibit a neuronal phenotype. The method involves exposing the cultured non-neuronal insect cells to an agent that impacts transcription of neuron-specific proteins, such as a steroidal insect hormone or a substance that mimics the activity of a steroidal insect hormone. In some embodiments of the invention, the transcriptional agent is administered to the cultured cells in combination with a mild depolarizing agent such as a cation channel agonist or antagonist. Exposure to these agents results in expression of one or more proteins associated with a neuronal phenotype (e.g. voltage-gated ion channels, ligand-gated receptors, neurotransmitter transporters and enzymes involved in neurotransmitter synthesis, turnover, and degradation). In addition, in some embodiments, expression of the proteins is also accompanied by the development of morphological features associated with a neuronal phenotype, e.g. with the development of axon-like structures and cell-to-cell contacts that resemble synapses. Such differentiated insect neuronal cells may be used for screening compounds such as candidate insecticides or drugs. The ability to induce a neuronal phenotype vastly simplifies the provision of insect neuronal cells, compared to either the use of primary cell culture or genetic engineering techniques.

The present invention provides a method for inducing a non-neuronal insect cell to exhibit a neuronal phenotype. The method includes the step of exposing the non-neuronal insect cell to an agent that regulates transcription of neuron-specific proteins in a quantity and for a time sufficient to induce the non-neuronal insect cell to exhibit a neuronal phenotype. In some embodiments, the agent that regulates transcription of neuron-specific proteins is a steroidal insect hormone or steroidal insect hormone mimic, for example, an ecdysone such as 20-hydroxyecdysone. In some embodiments, the method also comprises the step of exposing the non-neuronal insect cell to a mild depolarizing agent such as a cation channel agonist or antagonist, examples of which include but are not limited to sodium channel agonists, potassium channel antagonists, calcium channel agonists, and Na-K ATPase antagonists. In a preferred embodiment, the mild depolarizing agent is the sodium channel agonist veratridine. When the agent that regulates transcription of neuron-specific proteins is a steroidal insect hormone, it will be present in a quantity of 10-20 μg per ml of media in which said non-neuronal insect cell is grown. In a preferred embodiment of the invention, the neuronal phenotype is characterized by expression of neuron specific proteins, and/or the development of axon-like structures and putative synapses.

The present invention also provides a method for inducing a non-neuronal insect cell to exhibit a neuronal phenotype, which comprises the step of exposing the non-neuronal insect cell to an agent that regulates transcription of neuron-specific proteins in a quantity and for a time sufficient to induce the non-neuronal insect cell to exhibit a neuronal phenotype. In this method, the neuronal phenotype is characterized by expression of neuron specific proteins other than acetylcholinesterase. The method may also include-the-step of exposing the cultured non-neuronal insect cell to a mild depolarizing agent.

The invention also provides a cultured non-neuronal insect cell that exhibits a neuronal phenotype.

### BRIEF DESCRIPTION OF THE DRAWINGS

FIG. 1. Sf21 cells grown in complete Trichoplusia ni Medium-Formulation Hink (TNM-FH) medium.

FIGS. 2A and B. A, Sf21 cells in complete TNM-FH medium supplemented with fetal bovine serum and 20 μg/ml 20-hydroxyecdysone 48 hours after passage. B, Enlarged view of representative synapse formation (indicated by the arrow in A).

FIG. 3. Quantitative data from experiments similar to those shown in FIGS. 1 and 2. Sf21 cells displaying processes (“transformants”) were quantified in controls and those given 20-OH-ecdysone at 20 ug/ml. Bars are means with S.E. M. Asterisk indicates number of transformants is significantly different from control (cells without 20-OH-ecdysone) in a T-test, p<0.05.

FIG. 4. Quantitative data in Sf21 cells pre-conditioned by a 4 day incubation in 20-OH-ecdysone, then additionally exposed to DMSO, veratridine, or TTX. Initial numbers of transformants were subtracted to yield changes arising only from the second treatments. Bars are mean increase in transformants with S.E.M. Transformants were quantified in cells one day after DMSO alone (“D”-carrier for the veratridine at 1 ul/ml), flasks amended with 1 μM veratridine (“V”), or veratridine +1 μM tetrodotoxin (“V+TTX”). Asterisk indicates that the number of transformants in the E+V exposed group is significantly different from DMSO or those co-exposed to TTX (ANOVA, p<0.08).

FIG. 5. Sf21 cells in complete TNM-FH medium supplemented with 20 μg/ml nerve growth factor (NGF) 48 hours after passage.

### DETAILED DESCRIPTION OF THE PREFERRED EMBODIMENTS OF THE INVENTION

The present invention provides methods for inducing non-neuronal insect cells that are grown in culture to exhibit a neuronal phenotype. Induction of the neuronal phenotype results in patterns of protein expression in the cells that are characteristic of neuronal cells. For example, cells that are stimulated according to the methods of the present invention produce ion channels, neurotransmitter receptors, and other classes of protein targets that are of interest for insecticide and/or drug development efforts. In addition, morphological changes, such as the development of axon-like structures and cell-to-cell contacts that resemble synapses, may accompany the induction of neuronal protein expression.

The method involves exposing the non-neuronal insect cells to one or more agents that regulate transcription of neuron-specific proteins. In a preferred embodiment, the agent is a steroidal insect hormone or a compound that mimics the action of a steroidal insect hormone. In some embodiments of the invention, the transcriptional regulating agent is used in combination with one or more mild depolarizing agents.

By “neuronal phenotype” we mean that the induced insect cells express at least one protein that is specific to neurons at a level that is significantly higher than before induction. By “significantly higher” we mean that, prior to induction, the protein that is specific to neurons was undetectable by standard detection procedures (e.g. by antibody or activity assays), and that after induction the protein is detectable; or, alternatively, that after induction, the amount of the protein that is detected is at least about 2 fold higher than prior to induction, and preferably about 10 or more fold higher.

Examples of neuron-specific proteins that are induced by the methods of the present invention include but are not limited to various classes of ion channels that are involved in electrical excitability and are known to underlie the nerve membrane action potential. As a result, the effects of various compounds (e.g. insecticides) on those ion channels can be screened efficiently using the induced insect cultures. Examples of such voltage-sensitive ion channel classes include but are not limited to those involved in regulating the movement of sodium, calcium, potassium, and chloride ions, etc.

The induced insect cells of the present invention may also develop morphological changes associated with a neuronal phenotype. For example, the cells may develop long axon like processes and presumptive synapses, i.e. cell-to-cell contacts that resemble synapses. The formation of synapses allows screening of protein targets involved in both presynaptic and postsynaptic physiological mechanisms, such as ligand-gated receptors. Examples of ligand-gated receptors of interest include but are not limited to those recognized by substances such as: acetylcholine, amino acids, biogenic amines, and neuropeptides, etc.

Additional proteins involved in synaptic physiology that may be induced by the methods of the present invention include but are not limited to neurotransmitter transporters; enzymes involved in neurotransmitter synthesis, turnover, degradation, and other synaptic functions.

According to the method of the present invention, a neuronal phenotype is induced in cultured insect cells by supplementing the medium in which the cells are maintained with an agent that regulates transcription of proteins specific for insect neuronal cells. In a preferred embodiment of the invention, the agent is an insect steroidal hormone, or a compound that mimics the activity of an insect steroidal hormone. By “insect steroidal hormone” we mean a hormone that contains a four-ring steroid system. Examples of such hormones include but are not limited to various ecdysones such as ecdysone, 20-hydroxyecdysone, makisterone-A, 20-hydroxymakisterone-A. Compounds that mimic insect steroidal hormones are well known to those of ordinary skill in the art, and include, for example, ecdysone mimics such as tebufenozide.

Other agents that similarly impact transcriptional activity of neuron-specific proteins, either directly or indirectly, may also be used in the practice of the present invention. For example, pyrrolopyrimidines are known to influence transcription of neuronal cell proteins by blocking a kinase enzyme, which results in an increase in the cytoplasmic protein beta-catenin (Ding et al., 2003). As a result, beta-catenin is translocated into the nucleus of the cell where it impacts transcription, resulting in the expression of a neuronal phenotype in an otherwise uninduced cell.

The transcriptional impacting agents may be utilized in conjunction with mild depolarization agents in order to augment the effect of induction. Those of skill in the art are well acquainted with such agents, which include but are not limited to cation or anion channel agonists and antagonists that facilitate channel opening, prolong the open state of the channel or its single channel conductance, or prevent channel closure/inactivation. Exemplary compounds include but are not limited to: sodium channel agonists such as scorpion alpha and beta toxins, lipidoidal activators (e.g. veratridine), and pyrethroids or other sodium channel toxins such as ciguatoxin and brevatoxin; potassium channel antagonists such as apamin and 4-aminopyrimidine; calcium channel agonists such as BAYK-8644 and maitotoxin; and NaK-ATPase antagonists such as ouabain. Anion channel agonists would include for example an avernectin, and an anion channel antagonist could be one of several compounds, including but not limited to various stilbenes (DIDS, SITS), NPPB [5-nitro-2-(3-phenylpropylamino)benzoic acid], or anthracene-9-carboxylic acid. The cells to be induced may be exposed to the mild depolarization agent at the same time as their exposure to the transcriptional-impacting agent. Alternatively, the mild depolarization agent may be added to the medium before or after exposure to the transcription agent. In a preferred embodiment, the mild depolarization agent is added to the medium concomitantly with the transcription agent.

In general, the insect cells that are to be induced to exhibit a neuronal phenotype are exposed to a transcriptional-impacting agent and a mild depolarizing agent by addition of the agents to culture medium in which the cells are maintained. In general, the passage history of the culture is not crucial, which is an advantage of the method of the invention. Preferably, addition of the hormone is carried out on a culture that is subconfluent and attached to a substrate, such as a cell culture vessel. However, the precise form of the culture is also not crucial and may be any that is known to those of skill in the art, for example, the cells may be adhered to the inside surface of a vessel in a monolayer, suspended in solution, or grown in a manner that allows three-dimensional growth of the culture, e.g. on three-dimensional “scaffolding”, etc.

Typically, the amount of transcriptional agent (e.g. steroid hormone or mimic) that is added to the culture medium is in the range of about 1 to about 100 μg/ml, and preferably in the range of about 10 to about 20 μg/ml. Further, the amount of mild depolarizing agent (e.g. cation channel agonist) that is added to the culture medium is in the range of about 0.001 to about 100 μM, and preferably in the range of about 0.10 to about 1 μM.

A neuronal phenotype is observed in the uninduced cells after exposure to the agent for a period of time ranging from about 24 hours to about 336 hours. The precise amount of time necessary to induce a neuronal phenotype may vary somewhat from case to case, e.g. depending on the type and precise origin of the insect cell that is being induced. Thereafter, the cells are typically maintained in the presence of the inducing agent while in use, although the inducing agents can be removed once differentiation is achieved.

During culturing of the insect cells, uniform conditions are generally be maintained for a period of time with respect to temperature (typically about 27° C.) without a need for special consideration regarding humidity, CO₂ and O₂ levels, exposure to light, etc., although this may vary depending, for example, on the type of insect cell that is used, or other factors. Further, as those of skill in the art will appreciate, the culture medium that is employed may vary, depending on the type of insect cell that is being induced. Generally, medium such as Trichoplusia ni Medium-Formulation Hink (TMFH), etc. may be used. The media may be supplemented, for example, with antibiotics (e.g. gentamycin, penicillin, streptomycin, etc.), and other substances such as fetal bovine serum, etc. as warranted for successful growth and maintenance of the culture. Those of skill in the art are, in general, well acquainted with suitable insect cell culture techniques.

Those of skill in the art will recognize that many varieties of insects exist from which cells may be obtained and induced to exhibit a neuronal phenotype by the methods of the present invention. Cells from any insect of interest may be used in the practice of the present invention. Further, the particular tissue of origin of the cell within the insect is not crucial. A “non-neuronal” cell may be from any part of an insect, including neuroblast cells, so long as the cells are not yet differentiated.

According to the methods of the present invention, the insect cells that are cultured in vitro and induced to exhibit a neuronal phenotype may originate from any insect cell line, and preferably one that is immortal and commercially available. Those of skill in the art will recognize that several sources of immortal insect cell lines exist from which suitable cultures can be obtained, such as the American Type Culture Collection. By “immortal cell line” we mean a cell line comprising cells that can be maintained indefinitely under laboratory culture conditions. Examples of such immortal cell lines include but are not limited to Sf9, Sf21, etc. Alternatively, primary insect cell cultures may also be utilized in the practice of the invention. Regardless of the source of the insect cell, it is, in general, the immortal nature of the inducible cell that confers the advantages of the present invention.

Those of skill in the art will recognize that the induced insect cells of the present invention may be used in a wide variety of endeavors, including but not limited to the development and analysis of insecticides and nematicides, e.g. for the analysis of the efficacy of a compound, to determine the amount of a compound that is necessary to have a desired effect, etc; for drug development, e.g. to screen candidate compounds for activity against the insect cells, etc. All of these and other related types of endeavors are greatly facilitated by the present invention, particularly with respect to high throughput screening techniques.

The invention also provides insect cells that have been induced to exhibit a neuronal phenotype by the methods of the invention. Such insect cells display characteristic hallmarks of an insect neuronal cell, including the expression of ion channels involved in electrogenesis, the hallmark of nerve cells. In addition, the induced insect cells typically exhibit morphology characteristic of neuronal cells, e.g. the growth of axons and synaptic connections between cells. In some embodiments of the invention, the induced cells produce neuron specific proteins other than acetycholinesterase.

### EXAMPLES

### Example 1

Cell Culture Methods

The Sf21 insect cell line, derived from pupal ovarian tissue of Spodoptera frugiperda, the fall army worm, was obtained from commercial sources. Other insect cell lines work equivalently. The cells are maintained at appropriate temperature (e.g., 27° C.) in humidified or non-humidified environments. The cell medium for the studies described here was complete TNM-FH (Trichoplusia ni Medium-Formulation Hink with 10% fetal bovine serum and antibiotic, such as 10 μg/ml gentamycin). Other insect media (with or without fetal bovine serum) that maintain cells and support proliferation can be substituted for TNM-FH. For normal passage, cells at confluency are sloughed from the surface of the flask and diluted 1:5 in a new flask. For experimental treatments, flasks that had been passed at 1:5 were held in the incubator for 4 hours to allow the cells to reattach. In treatment flasks, the medium was removed and replaced with complete TNM-FH supplemented with 20-hydroxyecdysone, at various concentrations (e.g., 10 μg/ml), 5 g/ml of various retinoids, or 10 μg/ml nerve growth factor (NGF). The flasks were then observed daily and photographed.

Effects of Culture Media on Cell Phenotype

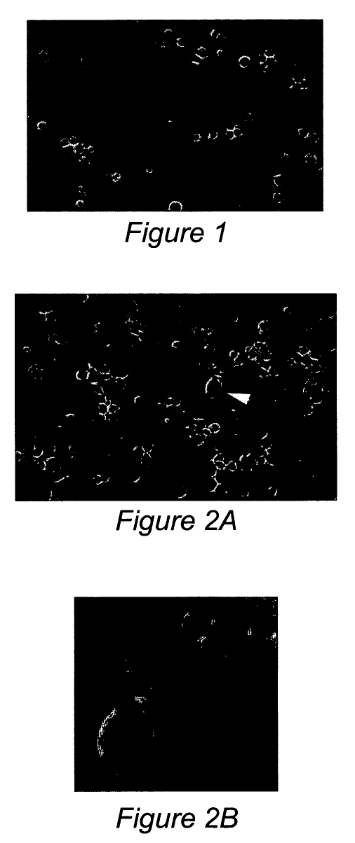

Sf 21 cells cultured in complete TNM-FH medium are shown in FIG. 1. The cells are uniform in size and spherical, with few cells showing elongation or branching cellular processes. FIG. 2 shows the effect of TNM-FH medium when supplemented with 10 μg/ml 20-hydroxyecdysone, 48 hours after passage. In the supplemented medium, numerous cells assume the appearance of monopolar neurons, with prominent axon-like structures. A smaller number of bipolar and multipolar neurons are also observed. In addition, numerous cell-to-cell contacts resembling synapses are observed. The white arrow in FIG. 2 identifies one of these contacts, which is enlarged to display the relevant structures in greater detail on the right hand side of the figure. The cultures stay in this differentiated state for a week or more with no further changes in the media.

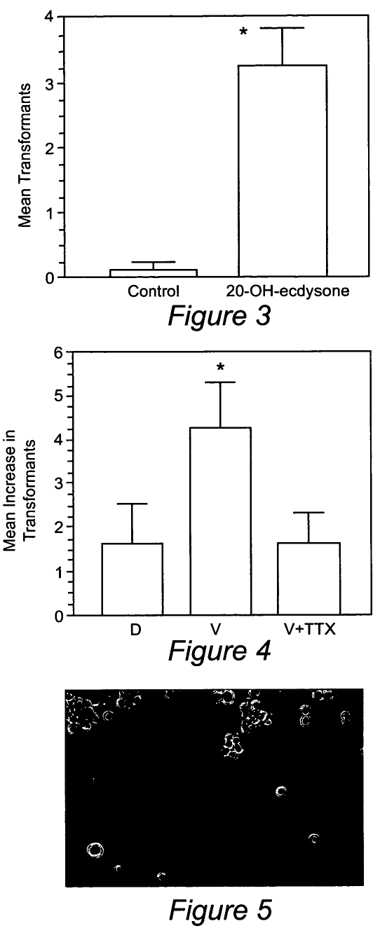

Efforts to quantify the transformation of Sf21 cells are displayed in FIG. 3. Transformed cells (those having processes, only rarely seen in controls) were counted 2 days after treatment with 20 μg/ml 20-hydroxyecdysone, similar to the micrographs shown in FIGS. 1 and 2. Culture flasks were scored on the outside lower surface with four intersecting lines prior to plating cells, and 4 counts were taken in a 1 square mm area at each intersection, on each of two plates exposed to each treatment, so that n=8 total. This procedure was used in all cases. There was about a 30-fold increase in the number of cells showing one or more processes following 20-OH-ecdysone exposure (FIG. 3).

The major ion channel underlying electrical activity in nerve cells is the voltage-sensitive sodium channel. This channel is activated by veratridine, blocked by tetrodotoxin (TTX), and is the conduit for sodium ions to enter the nerve cell during the upstroke of the action potential (Catterall, 1986). Further, a paper by Salthun-Lassalle (2004) found that neuronal cell cultures from rat embryos responded to a low dose of veratridine (1 μM) by increasing their survival, an effect that was blocked by co-incubation with 1 μM TTX. Incubation of cultures as described herein, pre-exposed for 4 days to 20-OH-ecdysone, produced a similar veratridine-protective effect (FIG. 4). Addition of 1 μM veratridine produced about a 3-fold increase in the number of transformants measured 24 hr after the veratridine treatment. This effect was blocked by the specific sodium channel blocker, TTX (FIG. 4). Thus, 20-OH-ecdysone elicits expression of endogenous sodium channels, the major ion channel involved in the nerve membrane action potential. Unlike mammals, sodium channels in insects are confined to the nervous system (Bloomquist, 1992; Song et al., 2004). Moreover, transformed insect cells respond to mild sodium channel activation in a manner indistinguishable from that of authentic neurons in culture. Thus, veratridine in conjunction with 20-OH-ecdysone forms an effective treatment for inducing a neuronal phenotype with optimized survival, and hence, utility.

Thus, the growth and differentiation of cells induced by this invention results in a culture having neuronal characteristics on both the cellular and tissue level. Growth of axons results in expression of voltage-dependent ion channels involved in electrical excitability. The axons within the nervous system carry information encoded as electrical impulses from cell to cell within the organism, similar to the axons and clumped cell bodies of these cultures. In addition, the formation of putative synapses in these cultures underlies the expression of proteins involved in presynaptic and postsynaptic mechanisms of cell-to-cell communication, similar to the neuropile of the central nervous system. The veratridine/TTX data demonstrates unambiguously that the major ion channel underlying neuronal excitation, the voltage sensitive sodium channel, is expressed following the treatment of cells according to the methods of the present invention.

FIG. 5 documents the observation that nerve growth factor does not induce any similar morphological changes in Sf21 cells. The cells continue to display a spherical shape with no processes or synaptic contacts. Further experiments with various isomers of retinoic acid also found no proliferative or differentiating effect. Rather, in most cases, a pronounced toxicity was observed when these compounds were tested at 10 μg/ml (data not shown).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

Andres A, and Cherbas, P. 1994. Tissue-specific regulation by ecdysone: distinct patterns of Eip28/29 expression are controlled by different ecdysone response elements. Dev. Genet. 15(4) p. 320-331.
Berger E, and Wyss C. 1980. Acetylcholinesterase induction by beta-ecdysone in Drosophila cell lines and their hybrids. Somatic Cell Genet. 6(5):631-40.
Best-Belpomme M, Courgeon A.-M, and Echalier G. 1980. Development of a model for the study of ecdysteroid action: Drosophila melanogaster cell established in vitro. In: Progress in Ecdysone Research; Developments in Endocrinology. J. A. Hoffman (ed.). Elsevier/North Holland Biomedical Press, Amsterdam, 379-392.
Camp et al., 1969. Journal of Agricultural and Food Chemistry 17: 243-248,
Catterall W. 1986. Molecular properties of voltage-sensitive sodium channels. Ann. Rev. Biochem. 55: 953-985.
Cherbas P, Cherbas L, Williams C M. 1977. Induction of acetylcholinesterase activity by beta-ecdysone in a Drosophila cell line. Science. 197(4300):275-7.
Cohen E. 1981. Acetylcholinesterase activity in an Aedes aegypti cell line. Experientia. 37(4): 429-31.
Decombel L, Smagghe G, and Tirry L. 2004. Action of major insecticide groups on insect cell lines of the Beet Armyworm Spodoptera exigua compared to larvicidal toxicity. In Vitro Cell. Dev. Biol.-Animal 40: 43-51.
Ding, S, Wu T, Brinker A, Peters E, Hur W, and Gray N. 2003. Synthetic small molecules that control stem cell fate. Proc. Natl. Acad. Sci. USA, 100: 7632-7637.
Grünewald B. and Levine R. 1998. Ecdysteroid control of ionic current development in Manduca sexta motoneurons. J. Neurobiol. 37: 211-223.
Kislev N, Segal I, and Edelman M. 1984. Ecdysteroids induce morphological changes in continuous cell lines of Lepidoptera. Roux's Arch. Dev. Biol. 193: 252-256.
Lynn, D and Oberlander H. 1981. The effect of cytoskeletal disrupting agents on the morphological response of a cloned Manduca sexta cell line to 20-hydroxy-ecdysone. Wilhelm Roux's Archives 190: 150-155.
Matsuda et al., Trends in Pharmacological Sciences Vol.22, pp. 573-580, 2001) Wegener S, Spindler-Barth M, Spindler K. 1996. A muscarinic acetylcholine receptor, present in the epithelial cell line from Chironomus tentans. Biol. Chem. 377(12): 819-24.
Salthun-Lassalle B, Hirsch E, Wolfart J, Ruberg M, and Michel P. 2004. Rescue of mesencephalic dopaminergic neurons in culture by low-level stimulation of voltage-gated sodium channels. J. Neurosci. 24(26): 5922-5930.
Song W, Liu Z, Tan J, Nomura Y, and Dong K. 2004. RNA editing generates tissue-specific sodium channels with distinct gating properties. J. Biol. Chem. 279: 32554-32561.
Wegener S, Spindler-Barth M, Spindler K. 1996. A muscarinic acetylcholine receptor, present in the epithelial cell line from Chironomus tentans. Biol. Chem. 377(12):819-24.

1. A method for inducing a non-neuronal insect cell to exhibit a neuronal phenotype, comprising the step of
exposing said non-neuronal insect cell to an agent that regulates transcription of neuron-specific proteins in a quantity and for a time sufficient to induce said non-neuronal insect cell to exhibit a neuronal phenotype.

2. The method of claim 1, wherein said agent that regulates transcription of neuron-specific proteins is a steroidal insect hormone or steroidal insect hormone mimic.

3. The method of claim 2, wherein said steroidal insect hormone is an ecdysone.

4. The method of claim 3, wherein said ecdysone is 20-hydroxyecdysone.

5. The method of claim 1, further comprising the step of exposing said non-neuronal insect cell to a mild depolarizing agent.

6. The method of claim 5, wherein said mild depolarizing agent is a cation or anion channel agonist or antagonist.

7. The method of claim 6, wherein said cation channel agonist or antagonist is selected from the group consisting of sodium channel agonists, potassium channel antagonists, calcium channel agonists, and Na-K ATPase antagonists.

8. The method of claim 7, wherein said sodium channel agonist is veratridine.

9. The method of claim 2, wherein said steroidal insect hormone is present in a quantity of 10-20 μg per ml of media in which said non-neuronal insect cell is grown.

10. The method of claim 1, wherein said neuronal phenotype is characterized by expression of neuron specific proteins.

11. The method of claim 1, wherein said neuronal phenotype is characterized by development of axon-like structures and putative synapses.

12. A method for inducing a non-neuronal insect cell to exhibit a neuronal phenotype, comprising the step of
exposing said non-neuronal insect cell to an agent that regulates transcription of neuron-specific proteins in a quantity and for a time sufficient to induce said non-neuronal insect cell to exhibit a neuronal phenotype, wherein said neuronal phenotype is characterized by expression of neuron specific proteins other than acetylcholinesterase.

13. The method of claim 12, wherein said cultured non-neuronal insect cell is also exposed to a mild depolarizing agent.

14. A cultured non-neuronal insect cell that exhibits a neuronal phenotype.
